(19) United States
(12) Patent Application Publication
(10) Pub. No.: US 2024/0398852 A1
(43) Pub. Date: Dec. 5, 2024

(54) NEGATIVELY CHARGED PARTICLES FOR THE TREATMENT OF INFLAMMATION-RELATED BURN INJURIES

(71) Applicant: onCour Pharma, Inc., Northbrook, IL (US)

(72) Inventor: Michael BOYNE, Northbrook, IL (US)

(21) Appl. No.: 18/695,943

(22) PCT Filed: Sep. 29, 2022
(86) PCT No.: PCT/US2022/077282

§ 371 Date: Mar. 27, 2024

Related U.S. Application Data
(60) Provisional application No. 63/249,933, filed on Sep. 29, 2021.

(51) Int. Cl. A61K 31/765; A61K 9/16; A61K 45/06; A61P 17/02; A61P 29/00; A61P 37/04

(52) U.S. Cl. CPC A61K 31/765; A61K 9/16; A61K 45/06; A61P 17/02; A61P 29/00; A61P 37/04

(57) ABSTRACT

The present disclosure provides methods of treating burn injuries or inflammation associated with burn injuries by administering negatively charged particles.

### CROSS-REFERENCE TO RELATED APPLICATIONS

This application claims priority to and the benefit of U.S. Provisional Patent Application Ser. No. 63/249,933 filed on Sep. 29, 2021, which is incorporated by reference for all purposes.

### FIELD OF THE INVENTION

The present disclosure generally relates to negatively charged particles for use in reducing or treating inflammation associated with a burn injury.

### BACKGROUND

Burn injuries resulting from thermal, chemical or radiation exposure represent a significant healthcare burden. Burn injuries result in significant tissue damage, heat-induced protein denaturation, ischemia and inflammation. Recovery from burn injuries depends on the size, area and depth of the injury. While superficial burns affecting the epidermis heal within 14 days with minimal scarring, deeper burns involving the reticular epidermis take 3 or more weeks to heal and leave behind significant scarring. Immediately after a burn injury, the innate immune system is activated triggering inflammation at the site of injury. This early inflammatory response is characterized by the activation of tissue resident macrophages and recruitment of circulating inflammatory cells to the site of injury. Activated tissue resident macrophages begin the process of recovery by initiating the production of extracellular matrix proteins and growth factors and activating fibroblasts. Among the inflammatory cells recruited to the site of injury, monocyte-derived macrophages are one of the first and accumulate within 24-48 hours. Once recruited, monocyte-derived macrophages mount an inflammatory response via the production of chemokines and soluble pro-inflammatory cytokines such as IL-1, IL-6, IL-12 and TNF-α. Additionally, increased exposure to infectious agents due to the breakdown of skin acts as a further stimulus that potentiates local inflammation.

While some inflammation is essential for wound healing after burn injuries, excessive inflammation is associated with poor healing, burn wound conversion and negative outcomes including sepsis and multi-organ dysfunction syndrome (MODS). Excessive inflammation, initially localized to the site of injury, has been observed to spread systemically resulting in the pathogenesis of Systemic Inflammatory Response Syndrome (SIRS) that is characterized by an increased risk of sepsis and organ failure. The initial pro-inflammatory response leading up to SIRS is followed by an anti-inflammatory response, called compensatory anti-inflammatory response syndrome (CARS). CARS results in an immunosuppressive state. During the CARS period, burn injury patients are at an increased risk of developing infections and multi-organ failure. While significant resources have been devoted to comprehensive burn wound care that is initiated 72 hours after a burn injury, there is a critical need for therapeutic interventions that address acute inflammation observed within the first 24-48 hours after injury. Current methods for ameliorating acute inflammation, through the use of broad immunosuppressants, are not ideal for treating burn injuries as broad immunosuppression in a burn injury patient will further complicate the recovery process.

Peripheral monocytes circulating in the blood can be classified into groups depending on expression of surface markers. Mature human tissue resident monocytes are understood to have a CD14LOCD16+ (mouse counterpart CX3CR1HICCR2−Gr1−) phenotype while Inflammatory monocytes exhibit a CD14+CD16− (mouse counterpart CX3CR1LOCCR2+Gr1+) phenotype.

Inflammatory monocytes recruited to sites of inflammation are known to be associated with numerous pathological processes. Following recruitment to sites of inflammation, Ly6C^{HI} monocyte-derived cells act as potent facilitators of inflammation. Ly6C^{HI} monocytes differentiate into macrophages or dendritic cells which secrete cytokines, proteases and other soluble factors that promote inflammation, tissue damage, scarring and even death. Currently, there are no therapies that achieve targeted suppression of inflammatory monocytes without causing broad immunosuppression. Importantly, use of broad-acting steroids, non-steroidal anti-inflammatory agents or other therapeutics that provide brief relief from inflammation cause broad immunosuppression, and are not suitable for the treatment of burn injuries as they are likely to worsen recovery. Thus, there is a need for new treatments for burn injuries.

### SUMMARY

In one aspect, the present disclosure provides a method of treating a burn injury (e.g., inflammation associated with a burn injury) comprising administering negatively charged particles to a subject in need thereof. In some embodiments, the negatively charged particles have a negative zeta potential. In some embodiments, the negatively charged particles are free from attached peptide or antigenic moieties or other bioactive materials (i.e., additional therapeutic agent are not attached or embedded in the negatively charged particle). In some embodiments, the present disclosure provides a method of treating inflammation associated with a burn injury in a subject comprising administering negatively charged particles that are free from attached peptide or antigenic moieties or other bioactive materials (i.e., additional therapeutic agent are not attached or embedded in the negatively charged particle). In some embodiments, the negatively charged particles comprise polyglycolic acid (PLG), polylactic acid (PLA), a copolymer of PLG and PLA (poly(lactic-co-glycolic acid or PLGA), polystyrene, diamond, a liposome, PEG cyclodextran, iron, zinc, cadmium, gold, or silver, or combinations thereof. In some embodiments, the negatively charged particles are biodegradable polymers. In some embodiments, the negatively charged particles are polyglycolic acid (PLG), polylactic acid (PLA), a copolymer of PLG and PLA (poly(lactic-co-glycolic acid or PLGA), a liposome, or PEG cyclodextrin.

In some embodiments, the negatively charged particles comprise poly(lactic-co-glycolic acid) (PLGA). In some embodiments, the negatively charged particles comprise PLGA at a copolymer ratio of about 80:20, 50:50, 90:10 or 100:0 of polylactic acid:polyglycolic acid or polyglycolic acid:polylactic acid. In various embodiments, the negatively charged particles comprise about 50:50, about 80:20 to about 100:0 polylactic acid:polyglycolic acid or from about 50:50, about 80:20 to about 100:0 polyglycolic acid:polylactic acid. In various embodiments, the negatively charged particles comprise 50:50 polylactic acid:polyglycolic acid. In various embodiments, the negatively charged particles comprise polylactic acid:polyglycolic acid from about 99:1 to about 1:99, e.g., about 99:1, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, or about 1:99.

In various embodiments, the negatively charged particles have a zeta potential between −100 mV and −1 mV. In various embodiments, the negatively charged particles have a zeta potential between −80 mV and −30 mV. In some embodiments, the zeta potential of the negatively charged particles is from about −100 mV to about −40 mV, from about −75 mV to about −40 mV, from about −70 mV to about −30 mV, from about −60 mV to about −35 mV, or from about −50 mV to about −40 mV. In various embodiments, the zeta potential is about −30 mV, −35 mV, −40 mV, −45 mV, −50 mV, −55 mV, −60 mV, −65 mV, −70 mV, −75 mV, −80 mV, −85 mV, −90 mV, −95 mV or −100 mV, including all values and ranges that lie in between these values.

In some embodiments, the negative zeta potential is achieved by the presence of functional groups, such as carboxyl groups, on the particles' surface. In some embodiments, the negative zeta potential is achieved by surface functionalization. In various embodiments, the surface functionalization is achieved by carboxylation. In some embodiments, the carboxylation produces a negative charge on the particles. In some embodiments, the carboxylation increases the negative charge of the negatively charged particles.

In various embodiments, the diameter of the negatively charged particles ranges from 0.1 μm to 10 μm. In various embodiments, the negatively charged particles have an average diameter ranging from about 0.2 μm to about 2 μm; about 0.3 μm to about 5 μm; about 0.5 μm to about 3 μm; or about 0.5 μm to about 1 μm. In some embodiments, the negatively charged particles have a diameter of about 100 nm to 1500 nm, about 200 nm and 2000 nm, about 100 nm to 1000 nm, about 300 nm to 1000 nm, about 400 nm to 800 nm, or about 200 nm to 700 nm. In various embodiments, the negatively charged particles have an average diameter of about 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm, including all values and ranges that lie in between these values. In some embodiments, the diameter of the negatively charged particles is about 400 nm to about 800 nm.

In various embodiments, the negatively charged particles have a homogenous size distribution. In various embodiments, the negatively charged particles have a homogenous size distribution wherein at least 90% of the negatively charged particles have a diameter ranging from about 0.05 μm to about 10, about 0.1 μm to about 10 μm, about 0.1 μm to about 5 μm, about 0.1 μm to about 3 μm, about 0.3 μm to about 5 μm, or about 0.3 μm to about 3 μm. In various embodiments, the negatively charged particles have a homogenous size distribution wherein at least 90% of the negatively charged particles have a diameter of about 100 nm to 10000 nm, about 100 nm to 5000 nm, about 100 nm to 3000 nm, about 100 nm to 2000 nm, about 300 nm to 5000 nm, about 300 nm to 3000 nm, about 300 nm to 1000 nm, about 300 nm to 800 nm, about 400 nm to 800 nm, or about 200 nm to 700 nm. In various embodiments, the negatively charged particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the negatively charged particles have a homogenous size distribution wherein at least 50% of the negatively charged particles have a diameter ranging from about 0.05 μm to about 10 μm, about 0.1 μm and about 10 μm, about 0.1 μm and about 5 μm, about 0.1 μm and about 3 μm, about 0.3 μm and about 5 μm, or about 0.3 μm and about 3 μm.

In various embodiments, the negatively charged particles have a homogenous size distribution wherein at least 50% of the negatively charged particles have a diameter of about 100 nm to 10000 nm, about 100 nm to 5000 nm, about 100 nm to 3000 nm, about 100 nm to 2000 nm, about 300 nm to 5000 nm, about 300 nm to 3000 nm, about 300 nm to 1000 nm, about 300 nm to 800 nm, about 400 nm to 800 nm, or about 200 nm to 700 nm. In various embodiments, the negatively charged particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the negatively charged particles have a homogenous size distribution wherein at least 10% of the negatively charged particles have a diameter ranging from about 0.05 μm to about 10 μm, about 0.1 μm to about 10 μm, about 0.1 μm to about 5 μm, about 0.1 μm to about 3 μm, about 0.3 μm to about 5 μm, or about 0.3 μm to about 3 μm. In various embodiments, the negatively charged particles have a homogenous size distribution wherein at least 10% of the negatively charged particles have a diameter of about 100 nm to about 10000 nm, about 100 nm to about 5000 nm, about 100 nm to about 3000 nm, about 100 nm to about 2000 nm, about 300 nm to about 5000 nm, about 300 nm to about 3000 nm, about 300 nm to about 1000 nm, about 300 nm to about 800 nm, about 400 nm to about 800 nm, or about 200 nm to about 700 nm. In various embodiments, the negatively charged particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm.

In various embodiments, the negatively charged particles are PLGA negatively charged particles having a zeta potential ranging from about −80 mV to about −30 mV and a diameter ranging from about 200 nm to about 2000 nm. In various embodiments, the negatively charged particles are PLGA negatively charged particles having a zeta potential ranging from about −80 mV to about −30 mV and a diameter ranging from about 200 nm to about 2000 nm, optionally surface functionalized by carboxylation.

In various embodiments, the disclosure provides a method of treating burn injuries, or burn related injuries, in a subject comprising administering to the subject a composition comprising negatively charged PLGA negatively charged particles alone or in combination with a therapeutic, wherein said negatively charged particles do not comprise peptides, antigenic moieties or other bioactive agents and have a diameter ranging about 400 nm to 800 nm and a zeta potential ranging about −1 mV to about −100 mV, and wherein the subject has one or more burn-related injuries. In some embodiments, the burn injury is the result of an exposure to a thermal, chemical, radiation source, or combination thereof.

In various embodiments, the chemical source resulting in a burn injury is acid, alkali, oxidants, detergent, vesicants, phosphorous burn, metals, silicates (cement) or chemical injection injury. In some embodiments, the chemical source is sulfuric acid, nitric acid, hydrofluoric acid, hydrochloric acid, acetic acid, formic acid, phosphoric acid, phenols, chloracetic acid, sodium hydroxide, potassium hydroxide, calcium hydroxide, sodium hypochlorite, calcium hypochlorite, calcium chloride, chlorine gas, chlorine dioxide, ammonia, phosphates, sodium carbonate, lithium hydride, tricalcium silicate, dicalcium silicate, tricalcium aluminate, tetra-calcium aluminoferrite, peroxides, hydrogen peroxide, sodium percarbonate, peracetic acid, benzoyl peoride, ozone, potassium persulfate, potassium permanganate, potassium dichromate, sulfur dioxide, sodium dithionite, sodium borohydride, or sodium perborate.

In various embodiments, thermal burn is thermal contact burn, thermal radiation burns, thermal electrical burns, or fire burn. In various embodiments, thermal burn is from hot metals, hot liquids, scalds (wet heat), steam, grease, or flames (dry heat). In various embodiments, electrical burn is arc flash burn, lightning burn, electrical flame burn, or electrical circuit burn. Electrical injuries are due to high voltage, low voltage, alternating current, direct current, high ampere currents, or low ampere currents. In various embodiments, the radiation source is nuclear radiation, electromagnetic radiation, radiation from nuclear fission, lasers, ultraviolet (UV) radiation, X-ray radiation, gamma radiation, cosmic radiation (sunlight), ionizing radiation, non-ionizing radiation, alpha negatively charged particles, or beta negatively charged particles.

In various embodiments, the negatively charged particles are administered once daily, twice daily, three times per day, seven times per week, six times per week, five times per week, four times per week, three times per week, twice weekly, once weekly, once every two weeks, once every three weeks, once every 4 weeks, once every two months, once every three months, once every 6 months or once per year. In various embodiments, the negatively charged particles are administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks, or more.

In some embodiments, the negatively charged particles are administered intravenously, orally, nasally, intramuscularly, ocularly, transdermally, or subcutaneously.

In various embodiments, the subject is a mammal. In various embodiments, the subject is a human.

In various embodiments, the negatively charged particles are formulated in a composition comprising a pharmaceutical acceptable excipient.

Also contemplated is a composition comprising any of the negatively charged particles or compositions of the disclosure, or use thereof in preparation of a medicament, for treatment of any of the disorders described herein associated with burn injury. In some embodiments, the burn injury is the result of an exposure to a thermal, chemical, radiation source, or combination thereof.

The administration of the negatively charged particles improves one or more symptoms of burn injury. In some embodiments, the one or more symptoms are size of burn area in the subject, time to healing of the burn, change in skin thickness, tissue necrosis, swelling, edema, and levels of inflammatory cells at the injury site. In some embodiments, the administration reduces the size of the burn area by 1%, 2%, 5%, 10%, 20%, 30%, 40%, 50%, 60%, 70%, 80%, 90% or 100%, including all values and ranges that lie in between these values.

In some embodiments, the administration of the negatively charged particles reduces of the number of inflammatory monocytes, macrophages, granulocytes, and/or neutrophils at the site of burn injury. In some embodiments, the administration prevents the accumulation of potentially pathology causing neutrophils, monocytes and/or granulocytes at the site of the burn injury. In some embodiments the administration decreases the dermal inflammatory infiltrates at the site of the injury. In some embodiments, administration of the negatively charged particles decreases the levels of inflammatory metabolites.

In some embodiments, administration of the negatively charged particles increases the number of regulatory T-cells, regulatory myeloid cells, or anti-inflammatory dendritic cells, monocytes, macrophages at the site of the burn injury. In some embodiments, administration of the negatively charged particles increases the ratio of regulatory T-cells to effector T-cells. In some embodiments, administration of the negatively charged particles increases regeneration of damaged tissue in a subject with burn-related injuries. In some embodiments, administration of the negatively charged particles increases the level of anti-inflammatory metabolites.

In various embodiments, administration of the negatively charged particles alters protein levels in a subject. In various embodiments, the proteins are associated with an immune response, foreign body response, metabolism, apoptosis, cell death, necrosis, ferroptosis, autophagy, cell migration, endocytosis, phagocytosis, DNA damage, pinocytosis, tight-junction regulation, cell adhesion, differentiation, presence and/or absence of cell types, or combinations thereof. In various embodiments, the proteins are cytokines or chemokines. In various embodiments the proteins are cell signaling proteins. In various embodiments, the cytokines and chemokines are IL-1α, IL-1β, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1α, CXCL4 (MIP-1β, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TGF-β1, TGF-β2, TGF-β3, or combinations thereof. In various embodiments, the protein is a protease. In some embodiments, the administration reduces the level of protease activity at the site of the burn injury. In various embodiments, the protease is an aspartic protease, a cysteine protease, a metalloprotease, a serine protease, and/or a threonine protease. In various embodiments, the protease is ADAM1, ADAM2, ADAM7, ADAM8, ADAM9, ADAM10, ADAM11, ADAM12, ADAM15, ADAM17, ADAM18, ADAM19, ADAAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, or MMP28. In various embodiments, the protein is a tissue inhibitor of metalloproteinases. In various embodiments, proteins associated with apoptosis are P53, Caspase 1, Caspase 2, Caspase 3, Caspase 4, Caspase 5, Caspase 6, Caspase 7, Caspase 8, Caspase 9, Caspase 10, Caspase 11, Caspase 12, Caspase 13, Caspase 14, BCL-2, BCL-XL, MCL-1, CED-9, A1, BFL1, BAX, BAK, DIVA, BCL-XS, BIK, BIM, BAD, BID, or EGL-1. Several methods for assaying proteins from a biological sample have been described in the literature including enzyme-linked immunosorbent assay (ELISA), western blots, and mass spectrometry. In various embodiments the protein is one or more immunoglobulins (Ig). In various embodiments, the Ig is IgA, IgD, IgE, IgM, or a variant thereof. Several methods for the detection of immunoglobulins from a biological sample have been described in the literature including radioimmunoassay, ELISA, and ImmunoCap.

In some embodiments, administration of the negatively charged particles decreases the levels of inflammatory cytokines and chemokines by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the baseline determined from one or more biological samples collected prior to treatment with negatively charged particles.

In some embodiments, administration of the negatively charged particles increases the levels of anti-inflammatory cytokines and chemokines by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the baseline determined from one or more biological samples collected prior to treatment with negatively charged particles.

In some embodiments, administration of the negatively charged particles decreases the levels of inflammatory metabolites by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the baseline determined from one or more biological samples collected prior to treatment with negatively charged particles.

In some embodiments, administration of the negatively charged particles increases the levels of inflammatory metabolites by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the baseline determined from one or more biological samples collected prior to treatment with negatively charged particles.

In some embodiments, administration of the negatively charged particles increases the ratio of regulatory T-cells to effector T-cells. In some embodiments, the increase is 1.5 fold, 2 fold, 5 fold, 10 fold, 20 fold, 40 fold, 60 fold, 80 fold, or 100 fold including all values lying within this range.

In some embodiments, administration of the negatively charged particles decreases the levels of inflammatory protease activity by about 5%-100% (e.g about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the baseline determined from one or more biological samples collected prior to treatment with negatively charged particles.

In some embodiments, administration of the negatively charged particles increases the levels of regenerative protease activity by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) relative to the baseline determined from one or more biological samples collected prior to treatment with negatively charged particles.

In some embodiments, administration of the negatively charged particles increases skin regeneration, tissue regeneration, epithelialization, epidermal-stromal interactions, keratinocyte migration, growth factors, fibroplasia, angiogenesis, granulation tissue, collagen synthesis and/or deposition, extracellular matrix (ECM) formation, ECM remodeling, vascular maturation, vascular regression and/or scar tissue formation at the site of the injury.

In various embodiments, administration of the negatively charged particles increases the production of growth factors. In various embodiments the growth factor is adrenomedullin, angiopoietin, autocrine motility factor, bone morphogenetic proteins, ciliary neurotrophic factor, leukemia inhibitory factor, colony-stimulating factor, macrophage colony-stimulating factor, granulocyte colony-stimulating factor, granulocyte macrophage colony-stimulating factor, epidermal growth factor, ephrin A1, ephrin A2, ephrin A3, ephrin A4, ephrin A5, ephrin B1, ephrin B2, ephrin B3, erythropoietin, fibroblast growth factor, fibroblast growth factor 1, fibroblast growth factor 2, fibroblast growth factor 3, fibroblast growth factor 4, fibroblast growth factor 5, fibroblast growth factor 6, fibroblast growth factor 7, fibroblast growth factor 8, fibroblast growth factor 9, fibroblast growth factor 10, fibroblast growth factor 11, fibroblast growth factor 12, fibroblast growth factor 13, fibroblast growth factor 14, fibroblast growth factor 15, fibroblast growth factor 16, fibroblast growth factor 17, fibroblast growth factor 18, fibroblast growth factor 19, fibroblast growth factor 20, fibroblast growth factor 21, fibroblast growth factor 22, fibroblast growth factor 23, foetal bovine somatotrophin, glial cell line-derived neurotrophic factor, neurturin, persephin, artemin, growth differentiation factor-9, hepatocyte growth factor, hepatoma-derived growth factor, insulin-like growth factor-1, insulin-like growth factor-2, keratinocyte growth factor, migration-stimulating factor, macrophage-stimulating protein, myostatin, neuregulin 1, neuregulin 2, neuregulin 3, neuregulin 4, brain-derived neurotrophic factor, nerve growth factor, neurotrophin-3, neurotrophin-4, placental growth factor, platelet-derived growth factor, renalase, T-cell growth factor, thrombopoietin, transforming growth factors, transforming growth factor alpha, transforming growth factor beta, tumor necrosis factor-alpha, or vascular endothelial growth factor. In various embodiments, the cytokine is miscellaneous hematopoietins, Epo, Tpo, Flt-3L, SCF, M-CSF, or MSP.

Administering the negatively charged particles with a second agent or concomitant therapy useful to treat burn injury is also contemplated. In some embodiments, the second agent is an immunosuppressant, an immune modulating agent or an antibiotic. In some embodiments, the second agent is a device that aids the healing of the burn area. In some embodiments, the device is a dressing, cellular and tissue based product, skin substitute or biologic graft, collagen dressing product, growth factors or other biologic wound management product, negative pressure wound therapy system, oxygen therapy device, wound debridement device, extracorporeal shock wave therapy device, electrical therapy or electromagnetic therapy device, anti-adhesion product, debriding and cleansing agent, wound closure sealant and glues, gauze, bismuth-impregnated petroleum gauze, or bandage or combinations thereof. In various embodiments the second agent or concomitant therapy is administered prior to, simultaneously or subsequently after administration of the negatively charged particles.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the disclosure. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIGS. 1A-1B: BALB/c mice were treated topically with NM to induce burn-related skin inflammation. Mice were intravenously administered with negatively charged particles at 3 hours, 24 hours, 48 hours and 72 hours post NM application. Treatment with NM+negatively charged particles decreased the rate of change in skin thickness compared to control mice treated with NM+phosphate buffered saline (PBS) (FIG. 1A). Negatively charged particles treatment reduced skin edema and dermal inflammatory infiltrates as seen in FIG. 1B. (n.s=P>0.5; *=p≤0.05; **=p≤0.01; ****=p≤0.0001).

FIGS. 2A-2B: Negatively charged particles treatment decreased the remaining wound area, assessed on day 5, compared to NM+PBS control (FIG. 2A and FIG. 2B).

FIGS. 3A-3B: Negatively charged particles treatment reduces myeloid cell infiltration and increases regulatory T-cells in the skin. Treatment with NM+PBS resulted in increased infiltration in the number of inflammatory, and non-inflammatory monocytes, macrophages, and neutrophils in the treated skin compared to naive mice (FIG. 3A). Treatment with negatively charged particles reduced the number of inflammatory, and non-inflammatory monocytes and macrophages in the inflamed skin area (FIG. 3A). Therapeutic negatively charged particles treatment increased the number of CD4+ T cells associated with wound healing in the compared to control mice treated with NM+PBS. Treatment with NM+PBS decreased the number of CD4+ T cells associated with wound healing in the treated cells compared to naive mice (FIG. 3B). Treatment with negatively charged particles increased the number of CD4+ T cells associated with wound healing as well as the T cells associated with wound healing/Teffector ratio in the affected skin (FIG. 3B).

FIGS. 4A-4D: T cells and macrophages associated with wound healing induced by negatively charged particles reduce inflammation associated with burn-related inflammatory responses. Mice were treated with anti-CD25 antibody 96 and 24 hours before NM application, in order to deplete T cells associated with wound healing. At 3, 24, 48 and 72 hours after NM application negatively charged particles were administered intravenously. As controls, mice were treated with an isotype IgG antibody. Treatment with negatively charged particles decreased the rate of change in skin thickness in animals pre-treated with the IgG isotype control antibody. The effect was reversed when the animals were pre-treated with the anti-CD25 antibody depleting T cells associated with wound healing (FIG. 4A and FIG. 4B). Negatively charged particles treatment also increased the number of IL-10⁺TGF-β⁺ myeloid-cells associated with wound healing: inflammatory monocytes, pDCs, non-inflammatory monocytes, macrophages and mDCs (FIG. 4C and FIG. 4D).

### DETAILED DESCRIPTION

The present disclosure is based on Applicant's discovery of the effects of negatively charged particles in burn injury treatment. In some embodiments, the burn injury is a result of an exposure to a thermal, chemical and/or radiation source.

It has been found that negatively charged particles derived from polystyrene, nano-diamonds or biodegradable polymers such as poly(lactic-co-glycolic) and surface-functionalized by carboxylation have immunomodulatory properties. Negatively charged particles infusion results in their uptake by Ly6C^{HI} circulating inflammatory monocytes expressing the scavenger receptor macrophage receptor with collagenous structure (MARCO). Negatively charged particles uptake triggers the redirection of monocytes in circulation to the spleen where they undergo Caspase 3-mediated apoptosis. Importantly, systemic administration of negatively charged particles has resulted in the reduction of inflammation and disease pathologies associated with the recruitment of inflammatory monocytes to sites of inflammation in multiple animal models of acute inflammation. Due to their specific targeting of MARCO-expressing circulating inflammatory monocytes, negatively charged particles do not cause broad immunosuppression. Thus, targeted inhibition of circulating inflammatory monocytes and suppression of their recruitment to the sites of burn injuries, while leaving the mediators of beneficial inflammation intact, may be a novel approach to reduce pathologies associated with the injury.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range within acceptable degree of variation of a stated value (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

It is noted here that as used in this specification and the appended claims, the singular forms “a,” “an,” and “the” include plural reference unless the context clearly dictates otherwise.

The term “particle” or “negatively charged particles” refers to any non-tissue derived composition of matter, it may be a sphere or sphere-like entity, bead, or liposome. The term “negatively charged particles”, “negatively charged particles”, “immune modifying negatively charged particles” (also referred to as IMP), and “beads” are interchangeably used herein.

The term “surface-functionalized” as used herein refers to introduction of chemical functional groups to a surface of a negatively charged particles. Surface functionalized negatively charged particles may be prepared by free-radical copolymerization of hydrophobic monomers with carboxylic acids, phosphates, hydroxyls, sulfonates, phosphonates, and amine or ammonium groups, as well as other functional groups. General methods of making surface functionalized nanonegatively charged particles are described in, for example, Froimowicz et al., Curr Org. Chem 17:900-912, 2013.

The term “biodegradable” refers to a negatively charged particles comprising a polymer that may undergo degradation, for example, by a result of functional groups reacting with the water in the solution. The term “degradation” as used herein refers to becoming soluble, either by reduction of molecular weight or by conversion of hydrophobic groups to hydrophilic groups. Biodegradable negatively charged particles do not persist for long times in the body, and the time for complete degradation can be controlled. Biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include PLA, PGA, polypropylene sulfide, and PLGA polymers. Biocompatible but non-biodegradable materials such as iron (Fe), zinc (Zn), cadmium (Cd), gold (Au), or silver (Ag) may also be used in the negatively charged particles described herein. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

The term “negatively charged particles” as used herein refers to negatively charged particles which have been modified to possess a net surface charge that is less than zero. Zeta potential is the charge that develops at the interface between a solid surface and its liquid medium. “Negative zeta potential” refers to a negatively charged particles having a zeta potential of the negatively charged particles surface as represented in milli Volts (mV) and measured by an instrument known in the field to calculate zeta potential, e.g., a NanoBrook ZetaPlus zeta potential analyzer or Malvern Zetasizer.

The term, “carboxylated particles” or “carboxylated beads” or “carboxylated spheres” includes any particles that possesses, has been modified, or surface functionalized to have present sufficient carboxyl groups on the particles surface to provide a negative zeta potential as described herein. In some embodiments the carboxyl groups enhance phagocyte/monocyte uptake of the negatively charged particles from circulation, for instance through the interaction with scavenger receptors such as MARCO. Carboxylation of the negatively charged particles can be achieved using any compound which adds carboxyl groups, including, but not limited to, poly(ethylene-maleic anhydride) (PEMA). Carboxylation may also be achieved by using polymers with native carboxyl groups (e.g., PLGA) to form particles.

The term “subject” as used herein refers to a human or non-human animal, including a mammal or a primate, that is administered a negatively charged particles as described herein. Subjects can include animals such as dogs, cats, rats, mice, rabbits, horses, pigs, sheep, cattle, and humans and other primates.

The term “therapeutically effective amount” is used herein to indicate the amount of target-specific composition of the disclosure that is effective to ameliorate or lessen one or more symptoms or signs of the disease or disorder being treated.

The terms “treat”, “treated”, “treating” or “treatment”, as used with respect to methods herein refers to eliminating, reducing, suppressing or ameliorating, either temporarily or permanently, either partially or completely, a clinical symptom, manifestation or progression of an event, disease or condition.

The phrase “free from attached peptide or antigenic moieties or other bioactive materials” refers to negatively charged particles that do not comprise a peptide, antigenic moiety or bioactive material. Said differently, therapeutically active peptides, antigenic moieties, or bioactive active agents are not attached, embedded, or otherwise associated with the negatively charged particles described herein. This phrase is intended to distinguish negatively charged particles coupled with therapeutic agents from the negatively charged particles described herein, which are themselves therapeutic agents. Thus, the phrase “free from attached peptide or antigenic moieties or other bioactive materials” is interchangeably used with the phrase “free from a therapeutic agent” or “free from an additional therapeutic agent.”

The term “control” refers to an otherwise identical subject with a burn injury that is not treated with negatively charged particles described herein or baseline measurements in the subject.

### Negatively Charged Particles

In some embodiments, the present disclosure provides for use of compositions comprising negatively charged particles (particles with a negative zeta potential), that are free of associated antigens, peptides or other bioactive materials (free from additional therapeutic agents) when used in the treatment methods.

Negatively charged particles can be formed from a wide range of materials. In embodiments, the negatively charged particles comprise a material suitable for biological use. For example, negatively charged particles may be composed of glass, silica, polyesters of hydroxy carboxylic acids, polyanhydrides of dicarboxylic acids, or copolymers of hydroxy carboxylic acids and dicarboxylic acids and biocompatible metals. In various embodiments, the negatively charged particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, negatively charged particles can be quantum dots, or composed of quantum dots, such as quantum dot polystyrene negatively charged particles (Joumaa et al. (2006) Langmuir 22:1810-6). Negatively charged particles including mixtures of ester and anhydride bonds (e.g., copolymers of glycolic and sebacic acid) may also be employed. In embodiments, the negatively charged particles comprise biodegradable polymers. For example, negatively charged particles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), [rho]oly (lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties.

In some embodiments, the negatively charged particles are diamond, PLG, PLA, PGA, polypropylene sulfide, PLGA polymers, polystyrene, a liposome, PEG cyclodextrin, or metals such as iron (Fe), zinc (Zn), cadmium (Cd), gold (Au) or silver (Ag). In some embodiments, the negatively charged particles are biodegradable. In some embodiments, the biodegradable negatively charged particles are PLG, PLA, PGA, polypropylene sulfide, PLGA polymers, or a liposome. Biocompatible but non-biodegradable materials may also be used in the negatively charged particles described herein. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

The negatively charged particles of the disclosure can be manufactured by any means known in the art. Exemplary methods of manufacturing negatively charged particles include, but are not limited to, microemulsion polymerization, interfacial polymerization, precipitation polymerization, emulsion evaporation, emulsion diffusion, solvent displacement, and salting out (Astete and Sabliov, J. Biomater. Sci. Polymer Edn., 17:247-289 (2006)). Methods of making negatively charged particles contemplated herein are disclosed in U.S. Pat. No. 9,616,113 and International Patent Publication WO/2017/143346, incorporated by reference in their entireties. Manipulation of the manufacturing process for PLGA negatively charged particles can control negatively charged particles properties (e.g., size, size distribution, zeta potential, morphology, hydrophobicity/hydrophilicity, polypeptide entrapment, etc.). The size of the negatively charged particles is influenced by a number of factors including, but not limited to, the concentration of polymer, e.g., PLGA, the solvent used in the manufacture of the negatively charged particles, the nature of the organic phase, the surfactants used in manufacturing, the viscosity of the continuous and discontinuous phase, the nature of the solvent used, the temperature of the water used, sonication, evaporation rate, additives, shear stress, sterilization, and the nature of any encapsulated antigen or polypeptide.

In various embodiments, the negatively charged particles comprise polymers, copolymers, dendrimers, diamond nanonegatively charged particles, or polystyrene nanonegatively charged particles. In various embodiments, the negatively charged particles comprise poly(lactide-co-glycolide) (PLG), polylactic acid (PLA), polystyrene, copolymers of PLG and PLA, diamond, a liposome, PEG, cyclodextran, or metals, such as iron, zinc, cadmium, gold, silver, or any combinations thereof.

In various embodiments, the negatively charged particles are co-polymer having a molar ratio from about 50:50 or about 80:20 to about 99:1 polylactic acid:polyglycolic acid or from about 50:50 or about 80:20 to about 99:1 polyglycolic acid:polylactic acid. In some embodiments, the negatively charged surface functionalized particle is a poly(lactic-co-glycolic acid) particle. In various embodiments, the negatively charged particles comprise 50:50 polylactic acid:polyglycolic acid. In various embodiments, the negatively charged particles comprise polylactic acid:polyglycolic acid from about 99:1 to about 1:99, e.g., about 99:1, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, and about 1:99, including all values and ranges that lie in between these values.

In some embodiments, the negatively charged particles have a zeta potential ranging from about −100 mV to about −1 mV. In some embodiments, the negatively charged particles have a zeta potential ranging from about −100 mV to about −50 mV, from about −80 mV to about −30 mV, from about −75 mV to about −50 mV, from about −70 mV to about −30 mV, from about −60 mV to about −35 mV, or from about −50 mV to about −40 mV, including any values and ranges therebetween. In various embodiments, the negatively charged particles have a zeta potential ranging from −30 mV, −35 mV, −40 mV, −45 mV, −50 mV, −55 mV, −60 mV, −65 mV, −70 mV, −75 mV, −80 mV, −85 mV, −90 mV, −95 mV or −100 mV, including any values and ranges therebetween.

In some embodiments, the negatively charged particles have an average diameter ranging from about 0.1 μm to about 10 μm. In some embodiments, the negatively charged particles have an average diameter ranging from about 0.2 μm to about 2 μm. In some embodiments, the negatively charged particles have an average diameter ranging from about 0.3 μm to about 5 μm. In some embodiments, the negatively charged particles have an average diameter ranging from about 0.5 μm to about 3 μm. In some embodiments, the negatively charged particles have an average diameter ranging from about 0.5 μm to about 1 μm. In some embodiments, the negatively charged particles have an average diameter of about 100 nm to 1500 nm, about 100 nm to 10000 nm, about 300 nm to 1000 nm, about 400 nm to 800 nm or about 200 nm to 700 nm, including any values or ranges therebetween. In various embodiments, the negatively charged particles have an average diameter of about 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm, including any values or ranges therebetween. In some embodiments, the negatively charged particles have an average diameter ranging from about 400 nm to about 800 nm.

In various embodiments, the negatively charged particles have a homogenous size distribution. In various embodiments, the negatively charged particles have a homogenous size distribution wherein at least 90% of the negatively charged particles have a diameter ranging from about 0.05 μm to about 10 μm, about 0.1 μm to about 10 μm, about 0.1 μm to about 5 μm, about 0.1 μm to about 3 μm, about 0.3 μm to about 5 μm, or about 0.3 μm to about 3 μm, including any values or ranges therebetween various embodiments, the negatively charged particles have a homogenous size distribution wherein at least 90% of the negatively charged particles have a diameter of about 100 nm to about 10000 nm, about 100 nm to about 5000 nm, about 100 nm to about 3000 nm, about 100 nm to about 2000 nm, about 300 nm to about 5000 nm, about 300 nm to about 3000 nm, about 300 nm to about 1000 nm, about 300 nm to about 800 nm, about 400 nm to about 800 nm, or about 200 nm to about 700 nm, including any values or ranges therebetween. In various embodiments, the negatively charged particles have a diameter of about 50 nm, about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, or about 2000 nm, or any values or ranges therebetween. In various embodiments, the negatively charged particles have a homogenous size distribution wherein at least 50% of the negatively charged particles have a diameter ranging from about 0.05 μm to about 10 μm, about 0.1 μm to about 10 μm, about 0.1 μm to about 5 μm, about 0.1 μm to about 3 μm, about 0.3 μm to about 5 μm, and about 0.3 μm to about 3 μm, including any values or ranges therebetween. In various embodiments, the negatively charged particles have a homogenous size distribution wherein at least 50% of the negatively charged particles have a diameter of about 100 nm to about 10000 nm, about 100 nm to about 5000 nm, about 100 nm to about 3000 nm, about 100 nm to about 2000 nm, about 300 nm to about 5000 nm, about 300 nm to about 3000 nm, about 300 nm to about 1000 nm, about 300 nm to about 800 nm, about 400 nm to about 800 nm, or about 200 nm to about 700 nm, including all values and ranges therebetween. In various embodiments, the negatively charged particles have a diameter of about 50 nm, about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, or about 2000 nm, including any values or ranges therebetween. In various embodiments, the negatively charged particles have a size distribution wherein at least 10% of the negatively charged particles have a diameter ranging from about 0.05 μm to about 10 μm, about 0.1 μm to about 10 μm, about 0.1 μm to about 5 μm, about 0.1 μm to about 3 μm, about 0.3 μm to about 5 μm, and about 0.3 μm to about 3 μm, including any values or ranges therebetween. In various embodiments, the negatively charged particles have a homogenous size distribution wherein at least 10% of the negatively charged particles have a diameter of about 100 nm to about 10000 nm, about 100 nm to about 5000 nm, about 100 nm to about 3000 nm, about 100 nm to about 2000 nm, about 300 nm to about 5000 nm, about 300 nm to about 3000 nm, about 300 nm to about 1000 nm, about 300 nm to about 800 nm, about 400 nm to about 800 nm, or about 200 to about 700 nm, including any values or ranges therebetween. In various embodiments, the negatively charged particles have a diameter of about 50 nm, about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, or about 2000 nm, including any values or ranges therebetween.

In some embodiments, the negatively charged particles are formulated in a sterile composition comprising one or more sterile pharmaceutically acceptable carriers. The phrase “pharmaceutically or pharmacologically acceptable” refers to molecular entities and compositions that do not produce allergic or other adverse reactions, or counter act the effects of the negatively charged particles, when administered to a subject as described below. The term “pharmaceutically acceptable carriers” includes any and all clinically useful solvents, dispersion media, coatings, antibacterial and antifungal agents, isotonic and absorption delaying agents and the like.

In some embodiments, the present disclosure provides a pharmaceutical composition comprising the negatively charged particles and a sterile pharmaceutically acceptable carrier or one or more additives. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant, and any combinations thereof. Examples of solutions, emulsions, or suitable carriers include aqueous or alcoholic/aqueous solutions, emulsions or suspensions, saline, and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers. A variety of aqueous carriers are suitable, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

In some embodiments, the negatively charged particles of the present disclosure are manufactured in a surfactant. In some embodiments, the negatively charged particles comprise a surfactant. In some embodiments, the surfactant is anionic, cationic, or nonionic. In some embodiments, the surfactants are poloxamer or poloaxamines. Other examples of surfactants include, but are not limited to, PEG, Tween-80, gelatin, dextran, pluronic L-63, PVA, methylcellulose, lecithin, DMAB, PEMA, and biodegradable and biocompatible surfactants such as vitamin E TPGS (D-α-tocopheryl polyethylene glycol 1000 succinate). In some embodiments, the negatively charged particles comprise multiple surfactants. For example, if the negatively charged particles is produced by a double emulsion method, the two surfactants can include a hydrophobic surfactant for the first emulsion, and a hydrophobic surfactant for the second emulsion.

In some embodiments, therapeutic formulations of the negatively charged particles may be prepared for storage by mixing the negatively charged particles having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, succinate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; or metal complexes (e.g., Zn-protein complexes).

In some embodiments, the negatively charged particles are stabilized by lyophilization. The addition of a cryoprotectant such as trehalose can decrease aggregation of the negatively charged particles upon lyophilization. Any suitable lyophilization and reconstitution techniques can be employed and adjusted to compensate any activity loss.

### Clinical Outcomes

In various embodiments, the current disclosure provides a method of reducing acute inflammation by redirecting circulating inflammatory monocytes to the spleen and inducing apoptosis in a subject comprising administering to the subject a pharmaceutical composition comprising negatively charged particles.

In various embodiments, the administration of the negatively charged particles in a subject reduces or prevents the accumulation of pathology causing monocytes, macrophages, granulocytes and/or neutrophils at the site of a burn injury, and/or reduces activation of said cells at the injury site.

In various embodiments, the current disclosure provides a method of reducing of the number of monocytes, macrophages, granulocytes and/or neutrophils at the site of a burn injury.

In various embodiments, the current disclosure provides a method of increasing the number of T-cells associated with wound healing, myeloid cells associated with wound healing, or non-inflammatory dendritic cells at the site of a burn injury. In various embodiments, the T-cells associated with wound healing are CD4+CD25+ T-cells. In various embodiments, the myeloid cells associated with wound healing are IL-10+ TGF-β⁺ cells.

In various embodiments, the administration improves one or more symptoms of burn injury. Exemplary symptoms include size of burn area in the subject, time to healing of the burn, tissue necrosis, swelling, edema, and levels of inflammatory cells at the injury site.

In some embodiments, the administration of the negatively charged particles reduces the burn area or by 10%, 20%, 30%, 40%, 50%, or more.

### Administration and Dosing

Contemplated herein are methods comprising administering a pharmaceutical composition comprising a negatively charged particles to treat a subject in need thereof.

Methods of the disclosure are performed using any medically-accepted means for introducing a therapeutic directly or indirectly into a mammalian subject, including but not limited to injections, oral ingestion, intranasal, topical, transdermal, parenteral, inhalation spray, vaginal, or rectal administration. The term parenteral as used herein includes subcutaneous, intravenous, intramuscular, intraarticular, intraperitoneal, intrathecal and intracisternal injections, as well as catheter or infusion techniques. In various embodiments, the negatively charged particles is administered intravenously, but may be administered through other routes of administration such as, but not limited to, intradermal, subcutaneous, epictuaneous, oral, intra-articular, and intrathecal. In some embodiment, the negatively charged particles is administered intravenously.

In various embodiments, the negatively charged particles are administered at a dose from about 0.1 mg/kg to about 10 mg/kg, from about 1 mg/kg to about 8 mg/kg, from about 1.5 mg/kg to about 10 mg/kg, from about 2 mg/kg to about 12 mg/kg, from about 2 mg/kg to about 10 mg/kg, from about 3 mg/kg to about 10 mg/kg, from about 4 mg/kg to about 10 mg/kg, from about 4 mg/kg to about 12 mg/kg, or from about 5 mg/kg to about 12 mg/kg, including any values or ranges therebetween. In embodiments, the negatively charged particles are administered in a dose of about 0.1 mg/kg, about 0.25 mg/kg, about 0.5 mg/kg, about 1.0 mg/kg, about 2.0 mg/kg, about 4.0 mg/kg, about 6.0 mg/kg, about 8.0 mg/kg, about 10 mg/kg, or about 12 mg/kg, or any values or ranges therebetween. In embodiments, the negatively charged particles are administered at a dose of about 1 mg, about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, or about 800 mg, including any values or ranges therebetween. In another embodiment, carrier particles are administered at a concentration ranging from about 0.05 mg/mL to about 50 mg/mL, for example, about 0.1 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12.5 mg/mL, about 15 mg/mL, about 17.5 mg/mL, about 20 mg/mL, about 25 mg/mL, about 30 mg/mL, about 40 mg/mL, or about 50 mg/mL, including any values or ranges therebetween. All values within and between the recited dose endpoints are contemplated.

In various embodiments, the negatively charged particles are administered within 24 hours after a burn injury (e.g., 1, 2, 3, 4, 5, 6, 7, 8, 9, 10, 11, 12, 13, 14, 15, 16, 17, 18, 19, 20, 21, 22, or 23 hours). In some embodiments, the negatively charged particles are administered to the subject within 48 hours after a burn injury. In some embodiments, the negatively charged particles are administered to the subject within 72 hours after a burn injury.

In various embodiments, the negatively charged particles are administered once daily, twice daily, three times per day, seven times per week, six times per week, five times per week, four times per week, three times per week, twice weekly, once weekly, once every two weeks, once every three weeks, once every 4 weeks, once every two months, once every three months, once every 6 months or once per year. In various embodiments, the negatively charged particles are administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks, or more.

### Combination Therapy

In some embodiments, the negatively charged particles of the disclosure are administered with a second agent useful to treat a burn injury. In some embodiments, the second agent is an immunosuppressant, an immune modulating agent, or an antibiotic. In some embodiments, the second agent is a device that aids in healing the burn area (e.g., a gauze, a bandage, and a hydrogel).

In various embodiments, the immunosuppressant is a steroid. In various embodiments, the steroid is beclomethasone, ciclesonide, fluticasone furoatr, mometasone, budenoside, fluticasone, triamcinolone, or loteprednol. In various embodiments, the immunosuppressant is a corticosteroid. In various embodiments, the corticosteroid is cortisone, prednisone, prednisolone, methylprednisolone, dexamethasone, betamethasone, or hydrocortisone.

In various embodiments, the additional therapeutic is a nonsteroid anti-inflammatory drug (NSAID). In various embodiments the NSAID is a non-selective NSAID. In various embodiments the NSAID is a COX-2 selective NSAID. In various embodiments the NSAID is a COX-1 selective NSAID. In various embodiments the NSAID is a prostaglandin synthase inhibitor. In various embodiments, the NSAID is diclofenac, diclofenac potassium, diclofenac sodium, diflunisal, etodolac, flurbiprofen, fenoprofen, fenoprofen calcium, ketorolac, ketorolac tromethamine, ketoprofen, tolmetin, tolmetin sodium, aspirin, ibuprofen, naproxen, indomethacin, indomethacin sodium, sulindac, felbinac, piroxicam, mefenamic acid, meclofenamate sodium, meloxicam, nabumetone, oxaprozin, piroxicam, celecoxib, etodolac, etoricoxib, lumiracoxib, rofecoxib, or valdecoxib.

In various embodiments, the immune modulating agent targets IL-6, IL-8, or TNF-α.

In various embodiments, the antibiotic is amoxicillin, doxycycline, cephalexin, ciprofloxacin, clindamycin, metronidazole, azithromycin, sulfamethoxazole, trimethoprim, levofloxacin, or any combination thereof. In various embodiments the antibiotic is a topical antibiotic. In various embodiments the topical antibiotic is silver sulfadiazine, polymyxin B, neomycin, erythromycin, mafenide acetate, chlorhexidine, povidone-iodine, sodium hypochlorite or bacitracin.

In various embodiments, the device that aids in healing the burn area is a dressing, cellular and tissue based product, skin substitute or biologic graft, collagen dressing product, growth factors and other biologic wound management product, negative pressure wound therapy system, oxygen therapy device, wound debridement device, extracorporeal shock wave therapy device, electrical therapy and electromagnetic therapy device, anti-adhesion product, debriding and cleansing agent, wound closure sealant and glues, gauze, bismuth-impregnated petroleum gauze, or bandage.

In various embodiments, the dressing is a foam dressing, hydrogel dressing, film dressing, alginate and gelling fiber dressing, hydrocolloid dressing, superabsorbent dressing, contact layer dressing, or composite dressing. Examples of foam dressing are polyurethane foam dressing, Hydrofiber foam dressing, Cutnova foam dressing, Lyofoam foam dressing, Covaderm tape foam dressing, hydrocellular dressing, sacral dressing, KerraFoam, Reston Self-Adhering Foam Dressing, Tegaderm Ag Mesh Dressing with Silver, Tegaderm Foam Adhesive Dressing, Tegaderm Silicone Foam Dressing, Tielle Essential Dressing, Askina Cavity Strips, Askina DresSil, Askina Foam, Askina Foam Cavity, Askina Heel, Askina Trachea, Biatain Adhesive, Biatain Fiber, Biatain Non-Adhesive, Biatain Silicone, Biatain Silicone Ag, Aquacel Ag Foam, Aquacel Foam, Aquacel Foam Pro, FoamLite. HydraFoam Ag, ComfortFoam, DermaBlue+, DermaFoam, DermaLevin, Optifoam, Lyofoam Max, Mepilex, Mepilex Border Flex, HydroTac, HydroTac Comfort, PermaFoam, PermaFoam Comfort, Proximel, Allevyn, Allevyn Ag, Allevyn Life, UrgoStart. Examples of hydrogel dressings are antibiotic-containing hydrogel, bioactive-agent containing hydrogel, hydrogel for skin substitution, propylene glycol containing hydrogel, cellulose hydrogel, hydroxymethylcellulose hydrogel, or carboxymethylcellulose hydrogel, Tegaderm Hydrocolloid Dressing, Kendall alginate hydrocolloid dressings, Comfeel, DuoDerm, Granuflex, GranuGel, DermaFilm, Exuderm, PrimaSeal Post Op Ag+, FlexiCol, Hydrocoll, Replicare, antibiotic containing hydrogel. Examples of superabsorbent dressing are Kerramax Care, Tegaderm Superabsorber Dressing, Biatain Super, ConvaMax, CovaWound, HydraLock SA, Xtrasorb, OptiLock, Qwick, RespoSorb, Zetuvit. Examples of contact layer dressing are Adaptic Touch, Tegaderm Non-Adherent Contact Layer, Askina SilNet Plus, Petrolatum Emulsion Contact Layer, Silicone Contact Layers, Biatain Contact, Physiotulle, ColActive Transfer, ComfiTel, SilverDerm 7, Dermanet Ag antimicrobial wound contact layer, Versatel, Mepitel, Atrauman, Atrauman Ag, Atrauman Silicone, Jelonet, Acticoat, Jelonet, UrgoStart Contact, UrgoTul, UrgoTul Ag/Silver, UrgoTul Silver Sulphadiazine (SSD). Other examples include Actisorb Plus, Actisorb Silver 220, Medipore+Pad Soft Cloth Adhesive Wound Dressing, Tegaderm Absorbent Clear Acrylic Dressing, Cardinal Health Composite Dressings, Granugel, DermaDress, Derma View II Island, Covaderm Plus, Transeal Plus, Iodosorb, Drawtex. Examples of gauze include plain gauze. Impregnated gauze, or fenestrated gauze.

In some embodiments, skin and tissue-based products, skin substitutes and/or biologic grafts include human cellular bioengineered grafts, bovine xenograft, porcine xenograft, equine xenograft, avian xenograft, piscine xenograft, autografts, or allografts. Examples of skin substitutes include AlloDerm, Neox, Biodesign Tissue Graft, AmnioExcel, AmnioExcel Plus, AmnioMatrix, Omnigraft Dermal Regeneration Template/Matrix, Kerecis Omega3 MicroGraft, AmnioBurn, AmnioCord, AmnioFix, EpiCord, EpiFix, TheraSkin, Therion, StrataGraft, Dermacyte Matrix, Affinity, Apligraf, Dermagraft, NuShield, TransCyte, Grafix, Stravix, Biovance, Interfyl, MySkin, Epicel, Bioskin. Examples of collagen dressing products include Fibrcol Plus Collagen Wound Dressing with Alginate, Cytal Wound Matrix, MicroMatrix, Promogran, Abccolla Collagen Matrix, Foam Calcium Alginate Topical Wound Dressing with Collagen, Foam Calcium Alginate Topical Wound Dressing, Aongen Collagen Matrix, Ologen Collagen Matrix, Endoform Topical Matrix, Myriad Negatively charged particles, Symphony, Awbat-S Awbat-D Awbat-M, Premvia, ProgenaMatrix, Fibrillar Collagen Wound Dressing, DuraMatrix, DuraMatrix Suturable, DuraMatrix-Onlay, DuraMatrix-Onlay Plus, Collawound Dressing, Woun'Dres Collagen Hydrogels, Cook ECM Powder (Oasis Micro), Oasis Wound Matrix, Coreleader Colla-Pad Model Cs 03030, ColActive Plus, ColActive Plus Ag, Collagen Wound Dressing, Healicoll, Scaffolene C1100 Bioresorbable Collagen Matrix, Geistlich Derma-Gide, Pelnac Bilayer Wound Matrix/TheraGenesis, Architect Px Extracellular Collagen Matrix, Bridge Extracellular Collegen Matrix, Collasorb Collagen Wound Dressing, Healadex-P Collatek Gel, Medifil II, Skintemp II, Hydrolyzed Collagen with 10% Chondroitin Sulfate (Polysulfated Glycosaminoglycan) Wound Gel hyCure, Integra Bilayer Wound Matrix, Integra Flowable Wound Matrix Model, Model: Fwd301, Integra Wound Matrix, Integra Wound Matrix (Thin), PriMatrix, PriMatrix Ag, Kerastat Cream, Kerastat Gel, Marigen Wound Dressing, Marigen Wound Extra, SurgiAid, Medline Collagen Wound Dressing, Puracol, Matriderm, Medtrade Products Alginate Island, Endoform Dermal Template, Bio-Connekt Wound Matrix, Neomatrix Wound Matrix, PuraPly AM, BioStep, BioStep AG, Stimulen Collagen, Xcellistem Wound Powder, Comatryx Collagen Wound Dressing, Apis, Unite Biomatrix, Excellagen, Innovamatrix, Innovamatrix Fs, XenoMem. Examples of growth factors and other biologic wound management products Cellutome, Aurix Autologel System, 3C Patch System, ActiGraft, Regranex, Examples of bandage include Kerlix dressings, Mepilex, Suprathel, Adaptic, Actociat, Ace Wraps, elastic wrap bandages, flexnet dressings, cotton batting, exudry dressings, kling dressings, soft kling dressings.

Examples of negative pressure wound therapy include Snap Therapy System, V.A.C.ULTA NPWT System, ALLY Therapy System, Catalyst, Pro Therapy System, SVED, Avelle, extriCare, UNO, XLR8, XLR8 Plus, Invia Liberty, Invia Motion, Avance, VivanoTec Pro, Pico Series, Renays, Venturi series of NPWT product range, VTG 190, VTG 2901, VTG 2901 V2, VTG 3900. Examples of oxygen therapy devices include Topical Wound Oxygen (TWO2), OxyGeni, O2Boot, O2Sacral, Natrox, Granulox, Epiflo, REZair. Examples of wound debridement devices include Qoustic Wound Therapy System, Jetox-ND, Irrisept, debritom+, SonicOne, UltraMist, Versajet II, and Pulsavac Plus. Examples of extracorporeal shock wave therapy devices include dermaPace, DermaGold. Examples of electrical stimulation and electromagnetic therapy devices include GV 350, Micro Plus, Diapulse. Examples of anti-adhesion products include 3M Wound Cleanser, Prontosan, Biolex Wound Cleanser, Adept Solution, Seprafilm, Adcon Gel, Sea-Clens Wound Cleanser, Sensi-Care, SAF AF Dermal Wound Cleanser, Curasalt, DermaKlenz, SafeWash, Interceed, Gentell Wound Cleanser, Dermagran Wound Cleanser, Primaderm Dermal Cleanser, Prophase, Mesalt, SilverMed Cleanser, Collagenase Santyl, Secura Cleanser, Repel-CV, Vashe. Examples of wound closure sealants and glues include Steri-Strip, Abra Surgical, LiquiBand, Histoacryl, Artiss, CoSeal, FloSeal, PreveLeak, TachoSil, Tisseel Fibrin Sealant, Progel, Sylys Surgical Sealant, BioGlue, StayStrips, Episeal, DuraSeal, Shur-Strip, Suture Strip Plus, Dermabond, SurgiFlo, Evicel, LiquiBand Rapid, Skin Affix, Leukostrip, Zip Surgical Skin Closure, TissuePatch, and WoundSeal Pour Pack Powder.

Concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks.

In some embodiments, the negatively charged particles and the second agent may be given simultaneously, in the same formulation. In some embodiments, the agents are administered in a separate formulation and administered concurrently, with concurrently referring to administering the agents on the same day.

In another aspect, the second agent is administered prior to administration of the negatively charged particle composition. Prior administration refers to administration of the second agent within the range of one week prior to treatment with the negatively charged particles, up to 30 minutes before administration of the negatively charged particles. In some embodiments, the second agent is administered subsequent to administration of the negatively charged particle composition. Subsequent administration is meant to describe administration from 30 minutes after negatively charged particles treatment up to one week after administration.

Additional aspects and details of the disclosure will be apparent from the following examples, which are intended to be illustrative rather than limiting.

### EXAMPLES

The following examples describe study designs to evaluate the safety, tolerability, pharmacodynamics, and efficacy of the negatively charged particles described in the present disclosure in burn injury or inflammation associated with burn injury treatment.

### Example 1

Mice models are used to analyze the effects of negatively charged particles on burn injury. Mice are administered with sulfuric acid (H₂SO₄), hydrochloric acid (HCl), sodium hydroxide (NaOH), lime (CaO), silver nitrate (AgNO₃), nitrogen mustard (NM) or hydrogen peroxide (H₂O2) to induce chemical burn, and exposed to thermal radiation, nuclear radiation, radio frequency energy, ultraviolet light or ionizing radiation to cause thermal burn or radiation burn, as described in Palmer et al., Developments of a Combined Radiation and Burn Injury Model, J. Burn Care Res., 32:317-323, 2011; Jang et al., In vivo Characterization of Early-Stage Radiation Skin Injury in a Mouse Model by Two-Photon Microscopy, Nature Scientific Reports, 6:19216, 1-9, 2016; Tewari-Singh et al., Inflammatory Biomarkers of Sulfur Mustard Analog 2-Chloroethyl Ethyl Sulfide-Induced Skin Injury in SKH-1 Hairless Mice, Toxicological Science, 108:192-306, 2009; Calum et al., Burn Mouse Models, Methods Mol. Biol., 1149:793-802, 2014; and Giles et al., A Peptide Inhibitor of c-Jun Promotes Wound Healing in a Mouse Full-Thickness Burn Model, Wound Repair and Regeneration, 16:58-64, 2008.

In this study, the negatively charged particles are administered to subject animals intravenously before and after induction of the sample burn and levels of inflammation related symptoms are measured, including inflammation related cytokines (IFNγ, TNFα, TGFβ) or reactive oxygen species (iNOS, nitric oxide, superoxide, hydrogen peroxide, myeloperoxidase), weight loss as a result of injury, and skin histology to determine the amount of inflammatory cells at the site of injury. Changes in the concentrations of inflammatory cytokines and reactive oxygen species, as well as reduced the number of inflammatory cells, such as monocytes, macrophages, granulocytes and/or neutrophils, at the site of injury are measured following administration of the negatively charged particles.

### Example 2

It was hypothesized that administration of negatively charged particles as described herein reduce the inflammation and injury associated with a combination of chemical, radiation and thermal burns, an animal model described in Palmer et al., Developments of a Combined Radiation and Burn Injury Model, J. Burn Care Res., 32:317-323, 2011. This study is designed to investigate the effects of the negatively charged particles in inflammation associated with burn injury treatment.

Mice are subjected to a 0-, 2-, 4-, 5-, 6-, or 9-Gy whole-body dose of ionizing radiation by exposure to a ¹³⁷Cs source in a Gammacell 40 irradiator (MDS Nordion, Ottawa, ON, Canada). The dose rate of irradiation in the Gammacell is 95 cGy per minute. Sham (OGy) irradiation mice are placed into the irradiator for matched amounts of time but without exposure to the source. One hour after radiation injury, mice are anesthetized with a mixture of ketamine (100 mg/kg) and xylazine (10 mg/kg) intra-peritoneally, and their dorsal surfaces were shaved with animal clippers. Mice are then placed into a plastic template with an opening allowing 15% TBSA on their dorsum to be exposed. Full-thickness scald injury is achieved by immersing the animals in a 95° C. water bath for 7 seconds. Immediately after exposure to water, animals are dried to prevent any further scalding. Sham animals are anesthetized, shaved, and immersed in room temperature water. To compensate for fluid loss and prevent circulatory shock, all animals receive 1.0 ml of warmed 0.9% normal saline intraperitoneally after the burn injury. Body temperature is maintained by placing the cages on heating pads until animals recovered from anesthesia. Some groups of animals are monitored twice daily for survival, and others are sacrificed at early time points, e.g., 6, 24, or 48 hours, or late time points of 14 or 28 days post-injury for analysis of various indices of systemic damage.

The negatively charged particles are administered to subject animals intravenously before and/or after induction of the sample burn and levels of inflammation related symptoms are measured, including inflammation related cytokines (IFNγ, TNFα, TGFβ) or reactive oxygen species (iNOS, nitric oxide, superoxide, hydrogen peroxide, myeloperoxidase), weight loss as a result of injury, and skin histology, to determine the amount of inflammatory cells at the site of injury. Changes in concentrations of inflammatory cytokines and reactive oxygen species, as well as reduced the number of inflammatory cells, such as monocytes, macrophages, granulocytes and/or neutrophils at the site of injury are measured following administration of the negatively charged particles.

### Example 3

### Negatively Charged Particles Improved Clinical Symptoms of Burn Injuries

The efficacy of negatively charged particles at treatment of nitrogen mustard (NM) induced skin inflammation in a therapeutic mouse model was examined.

The negatively charged particles used in this study had an average size of 400 nm-800 nm and a zeta potential of between −30 mV and −80 mV.

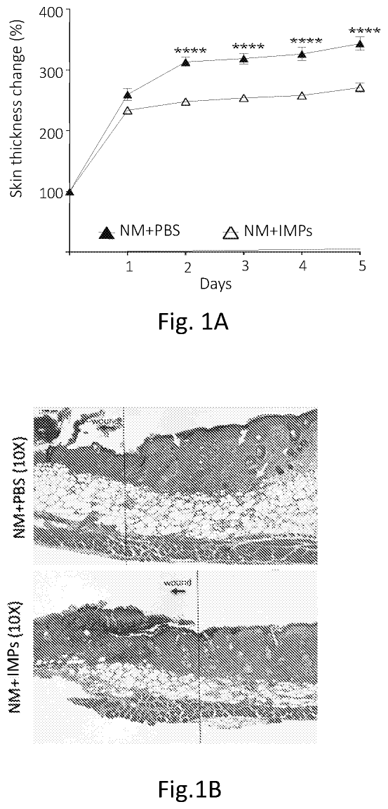

BALB/c mice were treated topically with NM to induce burn-related skin inflammation. Mice were intravenously administered with negatively charged particles at 3 hours, 24 hours, 48 hours and 72 hours post NM application. Treatment with NM+negatively charged particles decreased the rate of change in skin thickness compared to control mice treated with NM+phosphate buffered saline (PBS) (FIG. 1A). The negatively charged particles treatment reduced skin edema and dermal inflammatory infiltrates as seen in FIG. 1B.

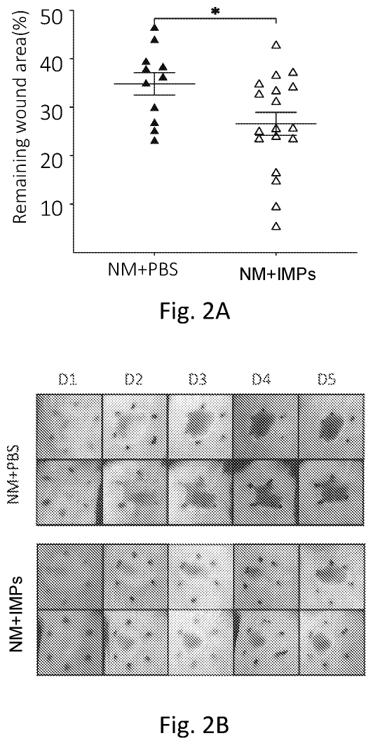

The negatively charged particles treatment decreased the remaining wound area, assessed on day 5, compared to NM+PBS control (FIG. 2A and FIG. 2B).

### Example 4

Negatively Charged Particles Reduced Myeloid Cell Infiltration and Increased T-Cells Associated with Wound Healing in the Skin

Treatment with negatively charged particles reduced the number of inflammatory (Ly6-C^{Hi}) and non-inflammatory (Ly6-C^{low}) monocytes recruited to the inflamed skin and a corresponding reduction in the number of macrophages in the inflamed skin.

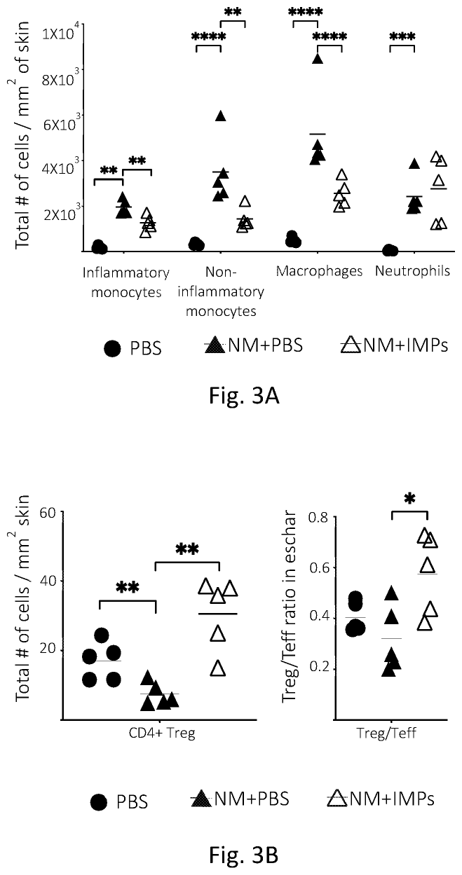

Therapeutic negatively charged particles treatment decreased the number of myeloid cells in the inflamed skin compared to control mice treated with NM+PBS. Untreated naive mice were used as controls for basal cell numbers. Treatment with NM+PBS resulted in increased infiltration in the number of inflammatory, and non-inflammatory monocytes, macrophages, and neutrophils in the treated skin compared to naive mice (FIG. 3A). Treatment with negatively charged particles reduced the number of inflammatory, and non-inflammatory monocytes and macrophages in the inflamed skin area (FIG. 3A).

Therapeutic negatively charged particles treatment increased the number of CD4+ T cells associated with wound healing in the compared to control mice treated with NM+PBS. Treatment with NM+PBS decreased the number of CD4+ T cells associated with wound healing in the treated cells compared to naive mice (FIG. 3B). Treatment with negatively charged particles increased the number of CD4+ T cells associated with wound healing as well as the T cell associated with wound healing to Teffector ratio in the affected skin (FIG. 3B).

These results indicate that negatively charged particles alleviate tissue destruction and inflammation in the burn-skin model by restricting myeloid cell infiltration and increasing the number of T-cells associated with wound healing in the inflamed skin.

### Example 5

T Cells Associated with Wound Healing and Macrophages Associated with Wound Healing Induced by Negatively Charged Particles Reduce Inflammation Associated with Burn-Related Inflammatory Responses

Mice were treated with anti-CD25 antibody 96 and 24 hours before NM application, in order to deplete T cells associated with wound healing. At 3, 24, 48 and 72 hours after NM application negatively charged particles were administered intravenously. As controls, mice were treated with an isotype IgG antibody.

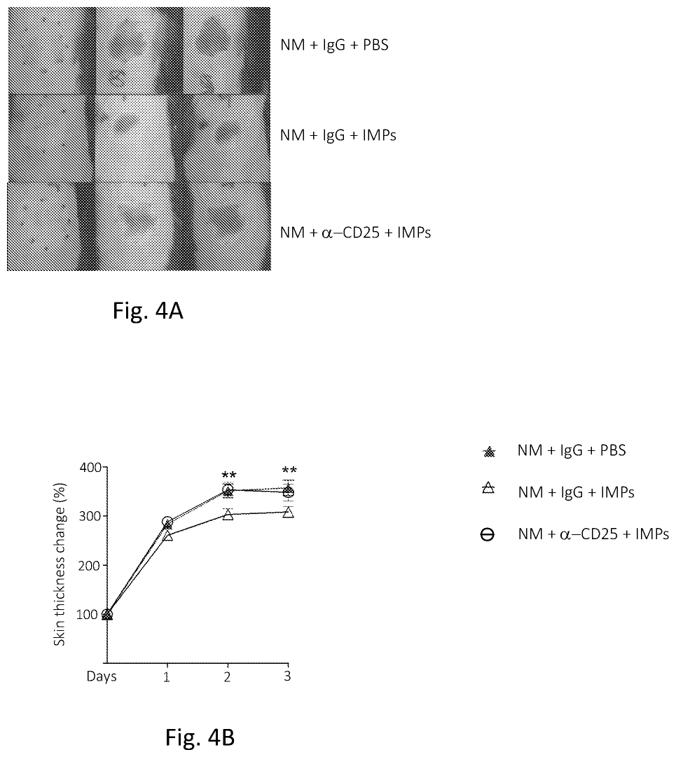

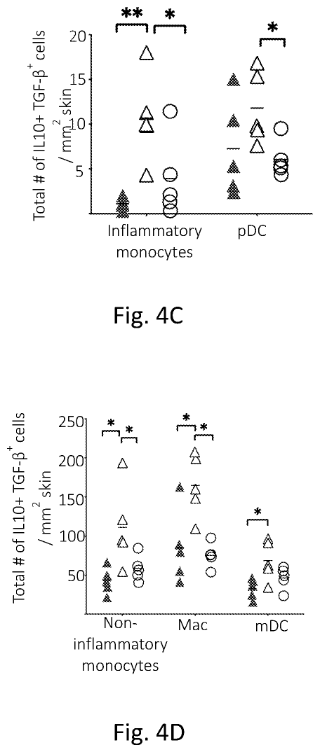

Treatment with negatively charged particles decreased the rate of change in skin thickness in animals pre-treated with the IgG isotype control antibody. The effect was reversed when the animals were pre-treated with the anti-CD25 antibody depleting T-cells associated with wound healing (FIG. 4A and FIG. 4B). Negatively charged particles treatment also increased the number of IL-10⁺TGF-β⁺ myeloid cells associated with wound healing: inflammatory monocytes, pDCs, non-inflammatory monocytes, macrophages and mDCs (FIG. 4C and FIG. 4D). These results indicate that T cells associated with wound healing and myeloid cells associated with wound healing induced by negatively charged particles reduce inflammatory responses caused by NM.

What is claimed is:

1. A method of treating inflammation associated with a burn injury in a subject in need thereof comprising administering to the subject negatively charged particles having a negative zeta potential, wherein the negatively charged particles free from attached peptide or antigenic moieties or other bioactive materials.

2. The method of claim 1, wherein the negatively charged particles comprise polyglycolic acid (PLG), polylactic acid (PLA), a copolymer of PLG and PLA (poly(lactic-co-glycolic acid or PLGA), polystyrene, diamond, a liposome, PEG cyclodextran, iron, zinc, cadmium, gold, or silver.

3. The method of claim 1 or 2, wherein the negatively charged particles comprise poly(lactic-co-glycolic acid) (PLGA).

4. The method of claim 3, wherein the negatively charged particles comprise PLGA at a copolymer ratio of about 90:10; 80:20, or 50:50 of polylactic acid:polyglycolic acid or polyglycolic acid:polylactic acid.

5. The method of any one of claims 1-4, wherein the negatively charged particles comprise PLGA with a copolymer ratio of about 50:50 of polylactic acid:polyglycolic acid.

6. The method of any one of claims 1-5, wherein the negatively charged particles have a zeta potential ranging from −100 mV to −1 mV

7. The method of any one of claims 1-6, wherein the negatively charged particles have a zeta potential ranging from −80 mV to −30 mV

8. The method of any one of claims 1-7, wherein the negative zeta potential is achieved by surface functionalization.

9. The method of any one of claims 1-8, wherein the negatively charged particles have an average size ranging from 100 and 1500 nm.

10. The method of any one of claims 1-9, wherein the negatively charged particles have an average size ranging from 100 and 1000 nm.

11. The method of any one of claims 1-10, wherein the negatively charged particles have an average size ranging from 400 and 800 nm.

12. The method of any one of claims 1-11, wherein the negatively charged particles are formulated in a composition comprising a pharmaceutically acceptable excipient.

13. The method of any one of claims 1-12, wherein the burn injury is a result of an exposure to a thermal, chemical, electrical, radiation source or combination thereof.

14. The method of any one of claims 1-13, wherein the chemical source resulting in a burn injury is acid, alkali, oxidants, detergent, vesicants, phosphorous burn, metals, silicates (cement) or chemical injection injury.

15. The method of claim 13, wherein the acid is sulfuric acid, nitric acid, hydrofluoric acid, hydrochloric acid, acetic acid, formic acid, phosphoric acid, phenols, chloracetic acid, sodium hydroxide, potassium hydroxide, calcium hydroxide, sodium hypochlorite, calcium hypochlorite, calcium chloride, chlorine gas, chlorine dioxide, ammonia, phosphates, sodium carbonate, lithium hydride, tricalcium silicate, dicalcium silicate, tricalcium aluminate, tetra-calcium aluminoferrite, peroxides, hydrogen peroxide, sodium percarbonate, peracetic acid, benzoyl peoride, ozone, potassium persulfate, potassium permanganate, potassium dichromate, sulfur dioxide, sodium dithionite, sodium borohydride, or sodium perborate.

16. The method of claim 13, wherein the thermal burn is thermal contact burn, thermal radiation burns, thermal electrical burns, or fire burn, is from hot metals, hot liquids, scalds (wet heat), steam, grease, or flames (dry heat),

17. The method of claim 13, wherein the electrical burn is arc flash burn, lightning burn, electrical flame burn, or electrical circuit burn, due to high voltage, low voltage, alternating current, direct current, high ampere currents, or low ampere currents.

18. The method of claim 13, wherein the radiation source is nuclear radiation, electromagnetic radiation, radiation from nuclear fission, lasers, ultraviolet (UV) radiation, X-ray radiation, gamma radiation, cosmic radiation (sunlight), ionizing radiation, non-ionizing radiation, alpha negatively charged particles, or beta negatively charged particles.

19. The method of any one of claims 1-18, wherein the negatively charged particles are administered once daily, twice daily, three times per day, seven times per week, six times per week, five times per week, four times per week, three times per week, twice weekly, once weekly, once every two weeks, once every three weeks, once every 4 weeks, once every two months, once every three months, once every 6 months or once per year.

20. The method of any one of claims 1-18, wherein the negatively charged particles is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks, or more.

21. The method of any one of claims 1-20, wherein the negatively charged particles are administered intravenously, orally, nasally, intramuscularly, ocularly, transdermally, or subcutaneously.

22. The method of any one of claims 1-21, wherein the subject is human

23. The method of any one of claims 1-22, wherein the administration improves one or more symptoms of burn injury.

24. The method of claim 23, wherein the one or more symptoms comprise reducing size of burn area in the subject, or reducing time to healing of the burn, change in skin thickness, tissue necrosis, swelling, edema, and levels of inflammatory cells at the injury site.

25. The method of claim 23 or 24, wherein the administration reduces the size of the burn area or by 5%, 10%, 20%, 30% or more compared to a control.

26. The method of claim 23 or 24, wherein the administration reduces of the number of inflammatory monocytes, macrophages, granulocytes, dermal inflammatory infiltrates, inflammatory metabolites and/or neutrophils at the site of burn injury compared to a control.

27. The method of claim 22 or 23, wherein the administration increases the number of regulatory T-cells, the number of regulatory myeloid cells, the number of non-inflammatory dendritic cells, the number of monocytes, the number of macrophages, the ratio of regulatory T-cells to effector T-cells, anti-inflammatory metabolites, regeneration of damaged tissue at the site of the burn injury compared to a control, or any combination thereof.

28. The method of any one of claims 1-27, wherein administration decreases inflammatory cytokines, chemokines, or both.

29. The method of claim 28, wherein the administration decreases inflammatory cytokines, chemokines, or both by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100 fold (e.g., about 2, 5, 10, 15, 20, 25, 30, 35, 40, 45, 50, 55, 60, 65, 70, 75, 80, 85, 90, 95, or 100-fold inclusive of all values and ranges between these values) compared to a control.

30. The method of claim 28, wherein the administration of the negatively charged particles increases anti-inflammatory cytokines and chemokines by about 5%-100% (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges between these values), 10-95%, 15-90%, 20-85%, 25-75%, 30-70%, 35-65%, 40-60%, 45-55%, or 50% or by about 2-100-fold (e.g., about 2, 5, 10, 15, 20, 25, 30, 35, 40, 45, 50, 55, 60, 65, 70, 75, 80, 85, 90, 95, or 100-fold inclusive of all values and ranges between these values) compared to a control.

31. The method of any one of claims 28-30, wherein the cytokines and/or chemokines are IL-1α, IL-1β, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1α, CXCL4 (MIP-1β, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TGF-β1, TGF-β2, TGF-β3, or combinations thereof.

32. The method of any one of claims 1-31, wherein the administration reduces the level of protease activity at the site of the burn injury.

33. The method of claim 32, wherein the protease comprises aspartic protease, cysteine protease, metalloprotease, serine protease, threonine protease, or any combination thereof.

34. The method of claim 32, wherein the protease comprises ADAM1, ADAM2, ADAM7, ADAM8, ADAM9, ADAM10, ADAM11, ADAM12, ADAM15, ADAM17, ADAM18, ADAM19, ADAAM20, ADAM21, ADAM22, ADAM23, ADAM28, ADAM29, ADAM30, ADAM33, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, or, MMP28, or any combination thereof.

35. The method of any one of claims 1-34, wherein the administration increases skin regeneration, tissue regeneration, epithelialization, epidermal-stromal interactions, keratinocyte migration, growth factors, fibroplasia, angiogenesis, granulation tissue, collagen synthesis and/or deposition, extracellular matrix (ECM) formation, ECM remodeling, vascular maturation, vascular regression, or scar tissue formation at the site of the injury, or any combination thereof.

36. The method of any one of claims 1-35, wherein the method further comprises administrating a second therapeutic agent useful to treat burn injury.

37. The method of claim 36, wherein the second agent is an immunosuppressant, an immune modulating agent, or an antibiotic.

38. The method of claim 37, wherein the immunosuppressant is a steroid, corticosteroid, or NSAID.

39. The method of claim 37, wherein the antibiotic comprises amoxicillin, doxycycline, cephalexin, ciprofloxacin, clindamycin, metronidazole, azithromycin, sulfamethoxazole, trimethoprim, levofloxacin, silver sulfadiazine, polymyxin B, neomycin, erythromycin, mafenide acetate, chlorhexidine, povidone-iodine, sodium hypochlorite or bacitracin.

40. The method of claim 36, wherein the second agent is a device that aids the healing of the burn area.

41. The method of claim 40, wherein the device is a dressing, cellular or tissue based product, skin substitute, biologic graft, collagen dressing product, growth factors, biologic wound management product, negative pressure wound therapy system, oxygen therapy device, wound debridement device, extracorporeal shock wave therapy device, electrical therapy, electromagnetic therapy device, anti-adhesion product, debriding or cleansing agent, wound closure sealant or glue, gauze, or bandage, or any combinations thereof.

42. The method of claim 41, wherein the dressing is a foam dressing, hydrogel dressing, film dressing, alginate and gelling fiber dressing, hydrocolloid dressing, superabsorbent dressing, contact layer dressing, composite dressing, polyurethane foam dressing, Hydrofiber foam dressing, Cutnova foam dressing, Lyofoam foam dressing, Covaderm tape foam dressing, hydrocellular dressing, sacral dressing, KerraFoam, Reston Self-Adhering Foam Dressing, Tegaderm Ag Mesh Dressing with Silver, Tegaderm Foam Adhesive Dressing, Tegaderm Silicone Foam Dressing, Tielle Essential Dressing, Askina Cavity Strips, Askina DresSil, Askina Foam, Askina Foam Cavity, Askina Heel, Askina Trachea, Biatain Adhesive, Biatain Fiber, Biatain Non-Adhesive, Biatain Silicone, Biatain Silicone Ag, Aquacel Ag Foam, Aquacel Foam, Aquacel Foam Pro, FoamLite, HydraFoam Ag, ComfortFoam, DermaBlue+, DermaFoam, DermaLevin, Optifoam, Lyofoam Max, Mepilex, Mepilex Border Flex, HydroTac, HydroTac Comfort, PermaFoam, PermaFoam Comfort, Proximel, Allevyn, Allevyn Ag, Allevyn Life, UrgoStart, Actisorb Plus, Actisorb Silver 220, Medipore+Pad Soft Cloth Adhesive Wound Dressing, Tegaderm Absorbent Clear Acrylic Dressing, Cardinal Health Composite Dressings, Granugel, DermaDress, Derma View II Island, Covaderm Plus, Transeal Plus, Iodosorb, Drawtex, or any combinations thereof.

43. The method of claim 42, wherein the dressing is a hydrogel dressing, antibiotic-containing hydrogel, bioactive-agent containing hydrogel, hydrogel for skin substitution, propylene glycol containing hydrogel, cellulose hydrogel, hydroxymethylcellulose hydrogel, carboxymethylcellulose hydrogel, Tegaderm Hydrocolloid Dressing, Kendall alginate hydrocolloid dressings, Comfeel, DuoDerm, Granuflex, GranuGel, DermaFilm, Exuderm, PrimaSeal Post Op Ag+, FlexiCol, Hydrocoll, Replicare, or antibiotic containing hydrogel, or any combinations thereof.

44. The method of claim 42, wherein the dressing is a superabsorbent dressing, Kerramax Care, Tegaderm Superabsorber Dressing, Biatain Super, ConvaMax, CovaWound, HydraLock SA, Xtrasorb, OptiLock, Qwick, RespoSorb, or Zetuvit or any combinations thereof.

45. The method of claim 42, wherein the dressing is a contact layer dressing, Adaptic Touch, Tegaderm Non-Adherent Contact Layer, Askina SilNet Plus, Petrolatum Emulsion Contact Layer, Silicone Contact Layers, Biatain Contact, Physiotulle, ColActive Transfer, ComfiTel, SilverDerm 7, Dermanet Ag antimicrobial wound contact layer, Versatel, Mepitel, Atrauman, Atrauman Ag, Atrauman Silicone, Jelonet, Acticoat, Jelonet, UrgoStart Contact, UrgoTul, UrgoTul Ag/Silver, or UrgoTul Silver Sulphadiazine (SSD), or any combination thereof.

46. The method of claim 41, wherein the gauze is plain gauze, impregnated gauze, bismuth-impregnated petroleum gauze, or fenestrated gauze, or combinations thereof.

47. The method of claim 41, wherein the biologic graft comprises human cellular bioengineered graft, bovine xenograft, porcine xenograft, equine xenograft, avian xenograft, piscine xenograft, autograft, or allograft, or a combination thereof.

48. The method of claim 47, wherein the biologic graft is AlloDerm, Neox, Biodesign Tissue Graft, AmnioExcel, AmnioExcel Plus, AmnioMatrix, Omnigraft Dermal Regeneration Template/Matrix, Kerecis Omega3 MicroGraft, AmnioBurn, AmnioCord, AmnioFix, EpiCord, EpiFix, TheraSkin, Therion, StrataGraft, Dermacyte Matrix, Affinity, Apligraf, Dermagraft, NuShield, TransCyte, Grafix, Stravix, Biovance, Interfyl, MySkin, Epicel, Bioskin.

49. The method of claim 41, wherein the collagen dressing product is Fibrcol Plus Collagen Wound Dressing with Alginate, Cytal Wound Matrix, MicroMatrix, Promogran, Abccolla Collagen Matrix, Foam Calcium Alginate Topical Wound Dressing with Collagen, Foam Calcium Alginate Topical Wound Dressing, Aongen Collagen Matrix, Ologen Collagen Matrix, Endoform Topical Matrix, Myriad Negatively charged particles, Symphony, Awbat-S Awbat-D Awbat-M, Premvia, ProgenaMatrix, Fibrillar Collagen Wound Dressing, DuraMatrix, DuraMatrix Suturable, DuraMatrix-Onlay, DuraMatrix-Onlay Plus, Collawound Dressing, Woun'Dres Collagen Hydrogels, Cook ECM Powder (Oasis Micro), Oasis Wound Matrix, Coreleader Colla-Pad Model Cs 03030, ColActive Plus, ColActive Plus Ag, Collagen Wound Dressing, Healicoll, Scaffolene C1100 Bioresorbable Collagen Matrix, Geistlich Derma-Gide, Pelnac Bilayer Wound Matrix/TheraGenesis, Architect Px Extracellular Collagen Matrix, Bridge Extracellular Collegen Matrix, Collasorb Collagen Wound Dressing, Healadex-P Collatek Gel, Medifil II, Skintemp II, Hydrolyzed Collagen with 10% Chondroitin Sulfate (Polysulfated Glycosaminoglycan) Wound Gel hyCure, Integra Bilayer Wound Matrix, Integra Flowable Wound Matrix Model, Model: Fwd301, Integra Wound Matrix, Integra Wound Matrix (Thin), PriMatrix, PriMatrix Ag, Kerastat Cream, Kerastat Gel, Marigen Wound Dressing, Marigen Wound Extra, SurgiAid, Medline Collagen Wound Dressing, Puracol, Matriderm, Medtrade Products Alginate Island, Endoform Dermal Template, Bio-Connekt Wound Matrix, Neomatrix Wound Matrix, PuraPly AM, BioStep, BioStep AG, Stimulen Collagen, Xcellistem Wound Powder, Comatryx Collagen Wound Dressing, Apis, Unite Biomatrix, Excellagen, Innovamatrix, Innovamatrix Fs, or XenoMem.

50. The method of claim 41, wherein the growth factor or biologic wound management product is Cellutome, Aurix Autologel System, 3C Patch System, ActiGraft, or Regranex.

51. The method of claim 41, wherein the bandage is Kerlix dressing, Mepilex, Suprathel, Adaptic, Actociat, Ace Wrap, elastic wrap bandage, flexnet dressing, cotton batting, exudry dressing, kling dressing, soft kling dressing.

52. The method of claim 41, wherein the negative pressure wound therapy device comprises Snap Therapy System, V.A.C.ULTA NPWT System, ALLY Therapy System, Catalyst, Pro Therapy System, SVED, Avelle, extriCare, UNO, XLR8, XLR8 Plus, Invia Liberty, Invia Motion, Avance, VivanoTec Pro, Pico Series, Renays, Venturi series of NPWT product range, VTG 190, VTG 2901, VTG 2901 V2, or VTG 3900, or any combinations thereof.

53. The method of claim 41, wherein the oxygen therapy device comprises Topical Wound Oxygen (TWO2), OxyGeni, O2Boot, O2Sacral, Natrox, Granulox, Epiflo, or REZair.

54. The method of claim 41, wherein the wound debridement device comprises Qoustic Wound Therapy System, Jetox-ND, Irrisept, debritom+, SonicOne, UltraMist, Versajet II, or Pulsavac Plus.

55. The method of claim 41, wherein the extracorporeal shock wave therapy devices comprise dermaPace or DermaGold.

56. The method of claim 41, wherein the electrical stimulation or electromagnetic therapy device comprises GV 350, Micro Plus, or Diapulse.

57. The method of claim 41, wherein the anti-adhesion device comprises 3M Wound Cleanser, Prontosan, Biolex Wound Cleanser, Adept Solution, Seprafilm, Adcon Gel, Sea-Clens Wound Cleanser, Sensi-Care, SAF AF Dermal Wound Cleanser, Curasalt, DermaKlenz, SafeWash, Interceed, Gentell Wound Cleanser, Dermagran Wound Cleanser, Primaderm Dermal Cleanser, Prophase, Mesalt, SilverMed Cleanser, Collagenase Santyl, Secura Cleanser, Repel-CV, or Vashe.

58. The method of claim 41, wherein the wound closure sealant or glue comprises Steri-Strip, Abra Surgical, LiquiBand, Histoacryl, Artiss, CoSeal, FloSeal, PreveLeak, TachoSil, Tisseel Fibrin Sealant, Progel, Sylys Surgical Sealant, BioGlue, StayStrips, Episeal, DuraSeal, Shur-Strip, Suture Strip Plus, Dermabond, SurgiFlo, Evicel, LiquiBand Rapid, Skin Affix, Leukostrip, Zip Surgical Skin Closure, TissuePatch, or WoundSeal Pour Pack Powder.

59. A method of treating a burn injury in a subject in need thereof comprising administering to the subject negatively charged particles comprising a biodegradable polymer, wherein the negatively charged particles free from a therapeutic agent, and the negatively charged particles have a zeta potential ranging from about −30 mV to about −80 mV.

60. The method of claim 59, wherein the negatively charged particles comprise poly(lactide-co-glycolide) (PLG), polylactic acid (PLA), a copolymer of PLG and PLA (poly(lactic-co-glycolic acid or PLGA), polystyrene, diamond, a liposome, PEG cyclodextran, iron, zinc, cadmium, gold, or silver.

61. The method of claim 59 or 60, wherein the negatively charged particles comprise PLGA.

62. The method of claim 61, wherein the PLGA comprises a copolymer ratio ranging from about 90:10 to 10:90 (e.g., 90:10; 80:20; 70:30; 60:40; 50:50; 40:60; 30:70; 20:80; or 10:90) of polylactic acid:polyglycolic acid.

63. The method of claim 61 or 62, wherein the PLGA comprises a copolymer ratio of about 50:50 of polylactic acid:polyglycolic acid.

64. The method of any one of claims 59-63, wherein the negatively charged particles have a zeta potential ranging from −70 mV to −30 mV or −60 mV to −30 mV.

65. The method of any one of claims 59-63, wherein the negatively charged particles have an average size ranging from 100 and 1500 nm.

66. The method of any one of claims 59-65, wherein the negatively charged particles have an average size ranging from 100 and 1000 nm.

67. The method of any one of claims 59-66, wherein the negatively charged particles have an average size ranging from 400 and 800 nm.

68. The method of any one of claims 59-67, wherein the negatively charged particles are formulated in a pharmaceutical composition comprising a pharmaceutically acceptable excipient.

69. The method of any one of claims 59-68, wherein the burn injury is a result of an exposure to a thermal source, chemical source, electrical source, radiation source, or any combination thereof.

70. The method of claim 69, wherein the chemical source is acid, alkali, oxidant, detergent, vesicant, phosphorous, metal, silicate (cement), or chemical injection injury.

71. The method of claim 70, wherein the acid is sulfuric acid, nitric acid, hydrofluoric acid, hydrochloric acid, acetic acid, formic acid, phosphoric acid, phenols, chloracetic acid, sodium hydroxide, potassium hydroxide, calcium hydroxide, sodium hypochlorite, calcium hypochlorite, calcium chloride, chlorine gas, chlorine dioxide, ammonia, phosphates, sodium carbonate, lithium hydride, tricalcium silicate, dicalcium silicate, tricalcium aluminate, tetra-calcium aluminoferrite, peroxide, hydrogen peroxide, sodium percarbonate, peracetic acid, benzoyl peoride, ozone, potassium persulfate, potassium permanganate, potassium dichromate, sulfur dioxide, sodium dithionite, sodium borohydride, or sodium perborate.

72. The method of claim 69, wherein the thermal burn is thermal contact burn, thermal radiation burn, thermal electrical burn, fire burn, or burn from hot metal, hot liquid, scald (wet heat), steam, grease, or flame (dry heat),

73. The method of claim 69, wherein the electrical burn is arc flash burn, lightning burn, electrical flame burn, or electrical circuit burn due to high voltage, low voltage, alternating current, direct current, high ampere currents, or low ampere currents.

74. The method of claim 69, wherein the radiation source is nuclear radiation, electromagnetic radiation, radiation from nuclear fission, laser radiation, ultraviolet (UV) radiation, X-ray radiation, gamma radiation, cosmic radiation (sunlight), ionizing radiation, non-ionizing radiation, radiation from alpha negatively charged particles, or radiation from beta negatively charged particles.

75. The method of any one of claims 59-74, wherein the negatively charged particles are administered once daily, twice daily, three times per day, seven times per week, six times per week, five times per week, four times per week, three times per week, twice weekly, once weekly, once every two weeks, once every three weeks, once every 4 weeks, once every two months, once every three months, once every 6 months or once per year.

76. The method of any one of claims 59-75, wherein the negatively charged particles are administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks, or more.

77. The method of any one of claims 59-76, wherein the negatively charged particles are administered intravenously, orally, nasally, intramuscularly, ocularly, transdermally, or subcutaneously.

78. The method of any one of claims 59-76, wherein the negatively charged particles are administered intravenously.

79. The method of any one of claims 59-78, wherein the subject is human.

80. The method of any one of claims 59-79, wherein the administration improves one or more symptoms of burn injury.

81. The method of claim 80, wherein the one or more symptoms comprise reducing size of burn area in the subject, reducing time to healing of the burn, change in skin thickness, tissue necrosis, swelling, edema, and levels of inflammatory cells at the injury site.
